(19) United States
(12) Patent Application Publication
(10) Pub. No.: US 2006/0014198 A1
(43) Pub. Date: Jan. 19, 2006

(54) Method for apparatus for detecting luminescence light from a porous support structure

(75) Inventors: Stephan Dertinger, Munich (DE); Marco Kluehr, Munich (DE)

Correspondence Address: DARBY & DARBY P.C., P. O. BOX 5257, NEW YORK, NY, 10150-5257, US

(73) Assignee: Infineon Technologies AG, Munich (DE)

(21) Appl. No.: 11/184,566

(22) Filed: Jul. 18, 2005

(30) Foreign Application Priority Data
Jul. 16, 2004 (DE) 10 2004 034 486.8

(51) Int. Cl. C12Q 1/68; G01N 33/53

(52) U.S. Cl. 435/6; 435/7.1

(57) ABSTRACT

Method and apparatus for detecting chemical and/or biochemical reactions and/or bindings. A two-dimensional support structure which has, distributed over at least one surface region, a multiplicity of pores which extend continuously from one surface of the support structure to the opposite surface, is provided wherein the pores are bounded by pore boundary areas of pore walls formed in the support structure, and the pore walls have a refractive index n_{pore wall} at a wavelength λ. A liquid is introduced into at least one of the pores of the support structure, where the refractive index n_{liquid} of the liquid at the wavelength λ is 0.90×n_{pore wall}>n_{liquid}≦1.10×n_{pore wall}. Light is coupling out of a substance to be investigated from the at least one pore, and the light of the substance to be investigated is detected.

### CROSS-REFERENCE TO RELATED APPLICATION

This application claims priority to German Patent Application Serial No. 10 2004 034 486.8, which was filed Jul. 16, 2004, and is incorporated herein by reference in its entirety.

### FIELD OF THE INVENTION

The present invention relates to a method for detecting chemical and/or biochemical reactions and/or bindings by means of detection of luminescence light from a porous support structure.

### BACKGROUND OF THE INVENTION

Biochips are now being used increasingly in molecular biology to obtain information about organisms and tissues in a rapid manner. The detection of (bio)chemical reactions, i.e. detection of biologically relevant molecules in defined investigation material, is of outstanding importance for the biosciences and medical diagnosis. Within this framework there are continuous advances in the development of so-called biochips. Such biochips are normally miniaturized hybrid functional elements having biological and technical components, in particular biomolecules which are immobilized on a surface of a biochip base module and serve as specific interaction partners. The structure of these functional elements frequently comprises rows and columns. The term then used is “microarrays”. Since thousands of biological or biochemical functional elements may be arranged on a chip, these are normally fabricated by microengineering methods.

Suitable biological and biochemical functional elements are, in particular, DNA, LNA, RNA, PNA, (in the case of nucleic acids and their chemical derivatives it is possible for example for single strands such as oligonucleotides, triplex structures or combinations thereof to be present), saccharides, peptides, proteins (e.g. antibodies, antigens, receptors), derivatives of combinatorial chemistry (e.g. organic molecules), cell constituents (e.g. organelles), single cells, multi-cellular organisms and cellular assemblages.

The most widely used variant of biochips are the so-called microarrays. These are small plates (“chips”) made of, for example, glass, gold, plastic or silicon. To detect appropriate biological or biochemical (binding) reactions, for example, small amounts of solubilized different capture molecules, e.g. a known nucleic acid sequence, are fixed in the form of very small droplets at points in a matrix, so-called dots, on the surface of the biochip base module.

In practice, some hundreds to some thousands of droplets are used per chip. Subsequently an analyte to be investigated, which may comprise for example fluorescence-labeled target molecules, is pumped over this surface. During this, generally various chemical (binding) reactions take place between the target molecules present in the analyte and the fixed or immobilized capture molecules. As already stated, the target molecules are labeled with dye molecule building blocks, normally fluorochromes, to observe these reactions or bindings. The presence and the intensity of light emitted by the fluorochromes provides information on the progress of the reaction or binding in the individual droplets on the substrate, so that conclusions can be drawn about the presence and/or the property of the target molecules and/or capture molecules. If the corresponding fluorescence-labeled target molecules of the analyte react with or bind to the capture molecules immobilized on the surface of the support substrate, this reaction or binding can be detected by optical excitation with a laser and measurement of the corresponding fluorescence signal.

Substrates with high but defined porosity have several advantages as basis for such biochips compared with planar substrates. More detection reactions can take place on the greatly enlarged surface area. This results in an increase in the sensitivity of detection for biological assays. The target molecules dissolved in the analyte are pumped through the channels between the front and rear sides of the porous substrate to bring them into close spatial contact with the surface of the substrate (<10 μm). At this scale of magnitude, diffusion is a very efficient transport process covering the distances between target molecule to be detected and the capture molecule immobilized on the surface within a short time. The speed of the binding reaction can thus be increased and therefore the duration of the detection method distinctly reduced.

An example of a substrate with such defined porosity is electrochemically produced porous silicon (cf. DE 42 02 454, EP 0 553 465 or DE 198 20 756).

A large proportion of the currently used analytical methods in drug research and clinical diagnosis makes use of optical methods for detecting binding events between substance to be detected and capture molecules (e.g. DNA hybridizations, antibody-antigen interactions and protein interactions). The substance to be detected is in these cases provided with a marker which, after excitation with light of suitable wavelength, fluoresces (fluorescence methods) or which induces a chemical reaction which in turn generates light (chemiluminescence methods). If the substance to be detected, i.e. the target molecule, binds to the immobilized capture molecule on the surface, optical detection, e.g. via luminescence, thereof is possible. The term “luminescence” refers in this connection to the spontaneous emission of photons in the ultraviolet to infrared spectral range. Luminescence-exciting mechanisms may be optical or non-optical in nature, for example electrical, chemical, biochemical and/or thermal excitation processes. It is thus intended that the term “luminescence” for the purposes of this invention includes in particular chemi-, bio- and electroluminescence plus fluorescence and phosphorescence.

Porous substrates with high optical density and low reflectivity, such as, for example, porous silicon, whose reflectivity in the visible range of the spectrum is from 50 to 70%, do not, however, provide the expected results in conjunction with fluorescence or chemiluminescence methods, in as much as the experimentally observed light signal yield is far removed from the theoretically achievable values. The reason for the experimentally observed light signal yield, which is reduced by comparison with the theoretical values, on use of such porous substrates is based, first on problems with the coupling out of the luminescence light of the substance or binding to be investigated, and secondly—in the case of a fluorescence method—on problems with the optical excitation of fluorescence.

WO03/089931A1 of the same applicant describes an apparatus which is suitable as “biochip base module”, and a detection method for chemical and biochemical reactions and/or bindings. For the luminescence light of the substance to be investigated to be efficiently coupled out of the porous support material, it was proposed to carry the luminescence light as waveguide mode in a waveguide structure in the support material. The efficiency of coupling out of the luminescence light which is brought about in this way leads to an increase in the sensitivity of detection.

### SUMMARY OF THE INVENTION

A method for detecting chemical and/or biochemical reactions and/or bindings. A two-dimensional support structure which has, distributed over at least one surface region, a multiplicity of pores which extend continuously from one surface of the support structure to the opposite surface, is provided, wherein the pores are bounded by pore boundary areas of pore walls formed in the support structure, and the pore walls have a refractive index n_{pore wall} at a wavelength λ. A liquid is introduced into at least one of the pores of the support structure, where the refractive index n_{liquid} of the liquid at the wavelength λ is 0.90×n_{pore wall}≦n_{liquid}≦1.10×n_{pore wall}. Light of a substance to be investigated is coupling out from the at least one pore, and the light of the substance to be investigated is detected.

### BRIEF DESCRIPTION OF THE DRAWINGS

The invention is described by way of example below with the aid of appended drawings of preferred embodiments, in which:

FIGS. 1(A) to (D) show diagrammatic sectional views of preferred support structures which are designed to be two-dimensional and have a multiplicity of pores into which a liquid comprising at least one substance to be investigated is introduced;

FIG. 2(A) shows a diagrammatic representation of excitation angle range and detection angle range for a preferred variant of the method of the invention for incident light dark-field excitation; and

FIG. 2(B) shows a diagrammatic view of the excitation angle and detection angle ranges for a preferred variant of a method of the invention for transmitted light dark-field excitation.

### DETAILED DESCRIPTION OF PREFERRED EMBODIMENTS OF THE INVENTION

It is an object of the invention to indicate a method for detecting chemical and/or biochemical reaction and/or bindings which, despite being simple to carry out, provides a high sensitivity of detection.

According to the invention, a method for detecting chemical and/or biochemical reactions and/or bindings includes the steps:

provision of a support structure which is designed to be two-dimensional and which has, distributed over at least one surface region, a multiplicity of pores which extend continuously from one surface of the support structure to the opposite surface,

where the pores are bounded by pore boundary areas from pore walls formed in the support structure, and

the pore walls have a refractive index n_{pore wall} at a wavelength λ;

introduction of a liquid comprising at least one substance to be investigated into at least one of the pores of the support structure, where the relation applying to the refractive index n_{liquid} of the liquid at the wavelength λ is 0.90×n_{pore wall}≦n_{liquid}≦1.10×n_{pore wall};

coupling out of light of the substance to be investigated from the at least one pore; and

detection of the light of the substance to be investigated.

The support structure is thus permeated by a multiplicity of pores which extend in the support structure and allow for example a liquid analyte to pass from one of the surfaces of the support structure to the opposite surface. The pores are bounded along their pore long axes by pore boundary areas which are formed in the pore walls in the support structure. The pore boundary areas thus represent the outer surfaces of the pore walls, i.e. the interfaces between the support material of the support structure and the pores to be filled.

By comparison with WO 03/089931A1, mentioned at the outset, the method of the invention for improving the sensitivity of detection for light from the substance to be investigated follows a fundamentally different approach, which is based on the following considerations.

In conventional support structures there is often a pronounced discontinuity of refractive index between the filled pores and the pore walls at the relevant wavelength, i.e. the luminescence wavelength to be detected). This discontinuity of refractive index causes, as “optical roughness” of the pore boundary areas, an extensive scattering both of the incident excitation light and of the light to be detected. Such a scattering of light in the porous support structure is particularly disadvantageous when the generally weak signals from binding reactions of molecules onto an immobilized “substance library” is to be detected via light. The scattering leads to an increased background signal which is composed of the light to be detected from the substance to be investigated and where appropriate of the scattered excitation light. In addition, the scattering also leads to a lower light signal because of scattering of the light at the pore boundary areas and increased absorption in the porous support structure. The signal-to-background difference is reduced thereby, implying a reduction in the sensitivity of detection of the method.

In the method of the invention, the pores of the porous support structure are filled with a liquid which has at a predefined wavelength λ a refractive index n_{liquid} which is in a range of ±10% of the refractive index n_{pore wall} of the pore wall adjoining the pore of the support structure at the predefined wavelength λ. The “leveling” of the refractive index difference |n_{pore wall}−n_{liquid}| between pore wall and liquid introduced into the pore effectively suppresses an unwanted scattering of excitation light or light at the pore boundary areas of the pore walls.

In other words, neither incident light nor emergent light which is to detected is reflected or scattered at the pore boundary areas due to the similar and preferably essentially identical refractive index of the pore wall compared with that of the liquid. If the support structure provides an immobilized “substance library”, and binding events are to be detected via a light signal, the reduction and preferably minimization of the refractive index difference leads to a reduction in the scattering of light in the support structure, making a higher absolute light signal achievable because less light is absorbed in the support structure.

The light from the substance to be investigated may be a light signal generated by absorption or reflection of excitation light, a luminescence light signal and/or a chemoluminescence light signal.

Detection of a light signal of the substance to be investigated, which is generated by absorption or reflection of an excitation light, is particularly advantageous for detection methods based on precipitation or staining. In these cases, in particular the result of a precipitation or staining reaction is obtained through detection of the light signal.

The substance to be investigated may be introduced into the pore in a preceding step of the method before filling the pore with the liquid. The substance to be investigated can have been introduced into the pore for example by means of a substance liquid which comprises the substance to be investigated, and is where appropriate at least partly bound to an immobilized “substance library” or capture molecules on the pore boundary areas. The substance to be investigated may also be the product of detection reactions of a substance which has been introduced into the at least one pore and is to be investigated.

The substance liquid is then removed from the pore. Before detection of light of the substance to be investigated, the liquid is passed into the at least one pore to which the relation 0.90×n_{pore wall}≦n_{liquid}≦1.10×n_{pore wall} applies.

This variant of the method of the invention is advantageous in particular for detection methods based on coupling out and detection of chemoluminescence. In this case, the liquid preferably comprises an appropriate chemoluminescence substrate for the substance to be investigated.

This variant of the method of the invention may also be advantageous for methods of the invention which are based on precipitation and staining methods.

However, it is likewise possible to introduce the substance to be investigated at the same time as the liquid to which the relation 0.90×n_{pore wall}≦n_{liquid}≦1.10×n_{pore wall} applies into the at least one pore, so that the preceding process step of introducing a substance liquid can be dispensed with.

Thus, for a preferred method of the invention based on coupling out and detection of a luminescence signal it may be advantageous initially to introduce the substance to be investigated into the at least one pore, subsequently to adjust the refractive index difference between pore wall and pore by introducing the liquid and subsequently to couple out and detect the luminescence signal. Alternatively, the introduction of the substance to be investigated and the adjustment of the refractive index difference can also take place simultaneously by introducing the liquid which comprises the substance to be investigated. Where appropriate it is not the introduced substance to be investigated but products of detection reactions of this substance which are investigated optically. In this case, these products represent the substance to be investigated.

For a preferred method of the invention based on a coupling out and detection of a chemoluminescence signal, it is preferred for initially the substance to be investigated to be introduced into the at least one pore and for the refractive index difference between pore wall and pore to be subsequently adjusted by introducing the liquid and simultaneously reading the chemoluminescence signal.

For a preferred method of the invention based on a precipitation or a staining (precipitation or staining reaction) it may be advantageous for there to be initial introduction of the substance to be investigated into the at least one pore, a subsequent development for carrying out the precipitation or staining reaction, a subsequent adjustment of the refractive index difference between pore wall and pore by introducing the liquid and subsequent coupling out and detection of the light signal for detecting the precipitation or staining reaction. The development step for carrying out the precipitation or staining reaction can also take place simultaneously with the adjustment of the refractive index difference.

The method of the invention is particularly suitable for detecting biochemical reaction and/or bindings, and for this purpose in particular for investigating enzymatic reactions, nucleic acid hybridizations, protein-protein interactions and protein-ligand interactions. The light to be detected may derive from reaction or binding events of the substance to be investigated with binding or reaction partners. A particular possibility is a fluorescence or chemoluminescence signal.

Preferably the relation 0.95×n_{pore wall}≦n_{liquid}≦1.05×n_{pore wall}, and preferably the relation 0.99×n_{pore wall}≦n_{liquid}≦1.01×n_{pore wall}, applies to the refractive index n_{liquid} of the liquid at the wavelength λ. It is particularly preferred for the refractive index n_{liquid} of the liquid at the wavelength λ to be essentially identical to the refractive index n_{pore wall} of the pore wall.

The support structure preferably consists at least in sections of a material whose refractive index at the wavelength λ is equal to n_{pore wall}. Excitation or detection light in such a section or compartment undergoes negligible scattering or refraction owing to the small or infinitesimal refractive index difference |n_{pore wall}−n_{liquid}| between support material and liquid, so that simple coupling in of excitation light and coupling out of light to be detected is possible. Different sections or compartments can be optically isolated from one another by opaque compartment boundaries.

In a preferred embodiment of the method of the invention, capture molecules are immobilized, at least in regions, where appropriate via linker molecules, on at least one of the pore boundary areas. The capture molecules are preferably selected from the group consisting of DNA, proteins and ligands. The capture molecules are preferably oligonucleotide probes which are bound via terminal amino or thiol groups to linker molecules which in turn are bound via covalent and/or ionic groups to the pore boundary area.

The linker molecules are normally based on a bifunctional organosilicon compound. Such bifunctional organosilicon compounds may be for example alkoxysilane compounds having one or more terminal functional groups selected from epoxy, glycidyl, chlorine, mercapto or amino. The alkoxysilane compound is preferably a glycidoxyalkylalkoxysilane such as, for example, 3-glycidoxypropyltrimethoxysilane, a mercaptoalkyl-alkoxysilane such as, for example, γ-mercaptopropyltrimethoxysilane, or an aminoalkylalkoxysilane such as, for example, N-β-(aminoethyl) γ-aminopropyltrimethoxysilane. The length of the alkylene radicals acting as spacer between the functional group such as, for example, epoxy or glycidoxy, and which binds to the actual capture molecule or the probe, and the trialkoxysilane group is subject to no restriction in this connection. Such spacers may also be polyethylene glycol residues. The oligonucleotides which can be used for example as capture molecules can be prepared by using the synthetic strategy as described in Tet. Let. 22, 1981, pages 1859 to 1862. The oligonucleotides can moreover be derivatized during the preparation method either at the 5 terminus or the 3 terminus with terminal amino groups. A further possibility for attaching such capture molecules can be carried out by initially treating the pore boundary areas or the first layer with a source of chlorine, such as Cl₂, SOCl₂, COCl₂ or (COCl)₂, where appropriate with use of a free-radical initiator such as peroxides, azo compounds or Bu₃SnH, and subsequently carrying out a reaction with an appropriate nucleophilic compound such as, in particular, with oligonucleotides or DNA molecules which have terminal primary amino groups or thiol groups (see WO 00/33976). It is thus possible to immobilize comprehensive “substance libraries” on the pore boundary areas.

In a further preferred embodiment, the support structure comprises silicon, silica and/or alumina. It is possible in particular to employ macroporous silicon structures. The design as glass microchannel plate is also preferred.

The liquid preferably comprises polyethylene glycol (PEG). It is particularly preferred to employ a mixture of at least two polyethylene glycols with different chain lengths, in which case it is possible to adjust the refractive index n_{liquid} by the ratio of mixing the two polyethylene glycols. For example, a mixture of polyethylene glycol with a molecular weight of 200 g/mol and polyethylene glycol with a molecular weight of 300 g/mol in the mixing ratio of 1/1 or 3/4 is advantageous for a partially oxidized silicon substrate (cf. in particular in relation to partially oxidized silicon substrates WO 03/089931 and WO 03/089925, which in this connection are an integral constituent of the disclosure of the present application). Such a PEG200/PEG300 mixture in the stated ratio has a refractive index in the relevant optical wavelength range which comes close to that of the partially oxidized silicon substrate.

In a further preferred embodiment, the liquid comprises water-soluble carbohydrates and derivatives thereof, especially a sucrose solution or an ethyl sorbate solution, where the refractive index n_{liquid} can preferably be adjusted via the concentration of the water-soluble carbohydrates. Particularly preferred for example for partially oxidized silicon substrates are 66 to 70% sucrose solutions, which have refractive indices of n=1.4558 to 1.4655. A 68.1% sugar solution whose refractive index in the relevant wavelength range is n_{liquid}=1.46085 and thus corresponds essentially to that of the partially oxidized silicon substrate (partOx substrate) is particularly suitable.

It is further possible and advantageous to use adhesives for fiber optics, medical technology and optical communication as liquid, it being possible to adjust the refractive indices thereof to the specific requirements. Optical adhesives available on the market typically have a refractive index of n_{liquid}=1.46 to 1.58.

It is additionally possible to use glycerol, whose refractive index n_{liquid}=1.4550, as liquid. It is also possible to employ silicones which are used for example as medical casting compositions. Organic substances such as, for example, dimethylformamide (refractive index n_{liquid}=1.42938) and heavy metal salts, especially thallium, can also be used. However, the compatibility thereof with “biochips” or microarray applications is only limited. It is generally necessary to take account of the dependence of the refractive index on the wavelength of the relevant light while adjusting the refractive index.

In a particularly preferred variant, the method of the invention includes the following further steps:

excitation of fluorescence light of the substance to be investigated by illumination of the substance to be investigated in the at least one pore along an excitation ray path with which excitation light impinges on the support structure, essentially at a predetermined excitation angle range from α to 180°−α and/or 180°+α to −α measured from a direction normal to the support structure which is designed to be two-dimensional;

detection of the excited fluorescence light of the substance to be investigated along a detection ray path with which fluorescence light is detected essentially at a predetermined detection angle range from −α to α and/or 180°−α to 180°+α measured from the normal direction.

In contrast to experimental constructions like those conventionally used for reading fluorescence signals from microarrays (imaging fluorescence microscopy and laser scanning microscopy), in the above particularly preferred embodiment of the method of the invention the excitation ray path and detection ray path do not run parallel to one another. Whereas conventionally the excitation ray path and detection ray path make use of the same lens system in order to illuminate the support structure and collect the fluorescence light, in the above embodiment of the invention there is a physical separation of the ray paths. The preferred illumination arrangement of the invention, which is similar to the classical dark-field illumination and with which excitation and emission paths are physically separated from one another, guarantees that excitation light cannot fall onto the detector, either directly or via reflections on surfaces, especially pore boundary areas, of the support structure. The geometric separation of excitation ray path and detection ray path in combination with the minimization of the refractive index difference between pore wall and liquid can make a further substantial reduction in the background signal, especially the reflected excitation light, on reading the support structure. The above variant is particularly suitable for microarrays based on partially oxidized silicon.

Besides the further minimization of the background signal, especially of the reflected excitation light, the preferred variant is distinguished by a mechanically robust and easily assembled optical design. Fluorescence excitation is easily possible in high-power LEDs with typical lateral emission characteristics so that simple and cost-effective portable readers with high detection sensitivity are possible with a low current consumption.

The “lateral” irradiation of the support structure with excitation light and the detection of the light essentially perpendicular to the support structure (i.e. in the direction normal thereto) via an aperture angle such that no direct light from the excitation ray path can enter the detection lens makes it possible further to improve the detection sensitivity.

The predetermined excitation angle range is preferably from α to 90° and/or 270° to 270°+α. This makes excitation as incident light dark-field excitation possible. Alternatively or in addition, the predetermined excitation angle range is from 90° to 180°−α and/or 180°+α to 270°, so that a transmitted light dark-field excitation is possible.

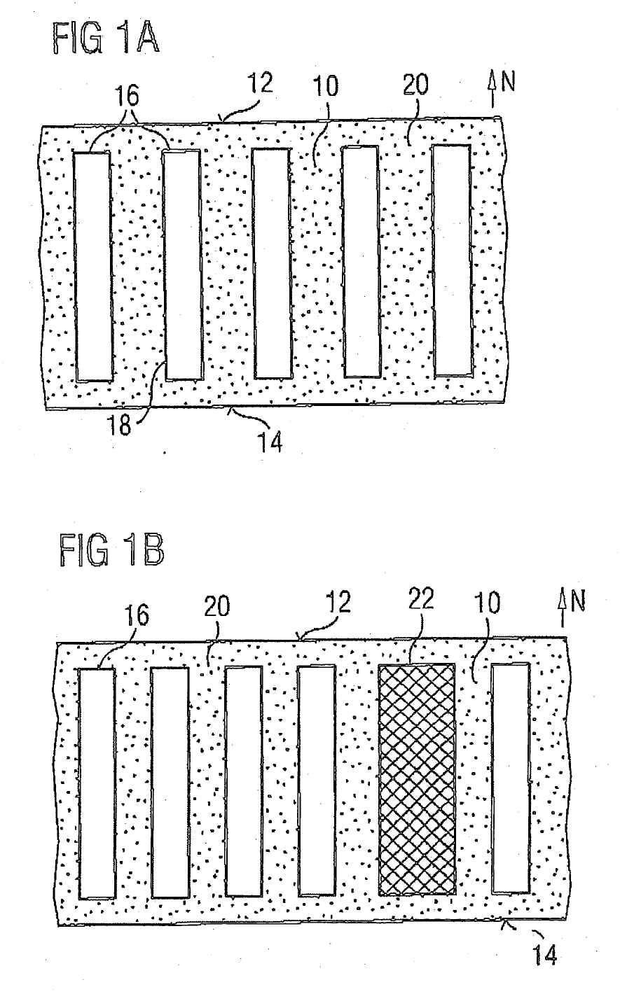

FIGS. 1(A) to (D) show highly diagrammatic sectional views through preferred support structures which are designed to be two-dimensional and which are suitable for a detection method of the invention. The section planes each run parallel to a normal direction N through the support structure. The support structure which is designed to be two-dimensional includes a multiplicity of pores 10 which extend continuously from one surface 12 to a surface 14 opposite to the support structure. Adjacent pores 10 are separated from one another by pore walls 16 whose outer surfaces facing the pore 10 are referred to as pore boundary areas 18. The refractive index n_{pore wall} of the pore wall 16 is preferably equal to or similar to the refractive index n_{liquid} of a diagrammatically depicted liquid 20 which comprises at least one substance to be investigated and which is introduced into the pores 10 of the support structure. The substance to be investigated may also have been introduced in a preceding step of the method into the at least one pore 10 and be immobilized at least partly on appropriate capture molecules on the pore boundary areas 18.

The minimization of the refractive index difference between pore wall 16 and the pore 10 filled with liquid 20 results in only a low or negligible optical scattering or refraction at the pore boundary areas 18, making it possible for excitation light and detection light respectively to be efficiently coupled in and out. This leads to an increase in the detection sensitivity for the (weak) luminescence signals.

In the embodiment depicted in FIG. 1(A), the pore walls 16 may consist for example of silicon dioxide. The pores 10 preferably have a pore diameter in the range from 500 nm to 100 μm. The support material preferably has a thickness in the normal direction N of from 100 to 5000 μm. The pore density of the pores 10 is preferably in the range from 10⁴ to 10⁸/cm².

FIG. 1(B) shows a further preferred support structure for a detection method of the invention. In the support structure shown in FIG. 1(B), a compartment partition 22 which consists of a material which is not optically transparent in the relevant wavelength range is additionally provided. The compartment partition 22 may comprise silicon for example. The compartment partition 22 optically separates two sections or compartments, each of which may comprise a multiplicity of pores 10, optically from one another.

FIG. 1(C) shows a further preferred support structure for a detection method of the invention. In this variant, the pore walls 16 are designed to be irregular in their surface configuration without, however, permitting connections between two adjacent pores 10. Suitable materials are once again for example macroporous silicon, porous silicon dioxide or alumina. FIG. 1(D) shows a further preferred support structure for a detection method of the invention, in which the pore walls 16 permit pore connections 24 between adjacent pores 10. Such pore walls 16 may be formed for example of Al₂O₃.

Suitable as liquid 20 are in principle all filling materials whose refractive index n_{liquid} can be adjusted to that of the porous support structure, shows no autofluorescence and with whose detection reaction is compatible in the support structure carrying in particular one substance library.

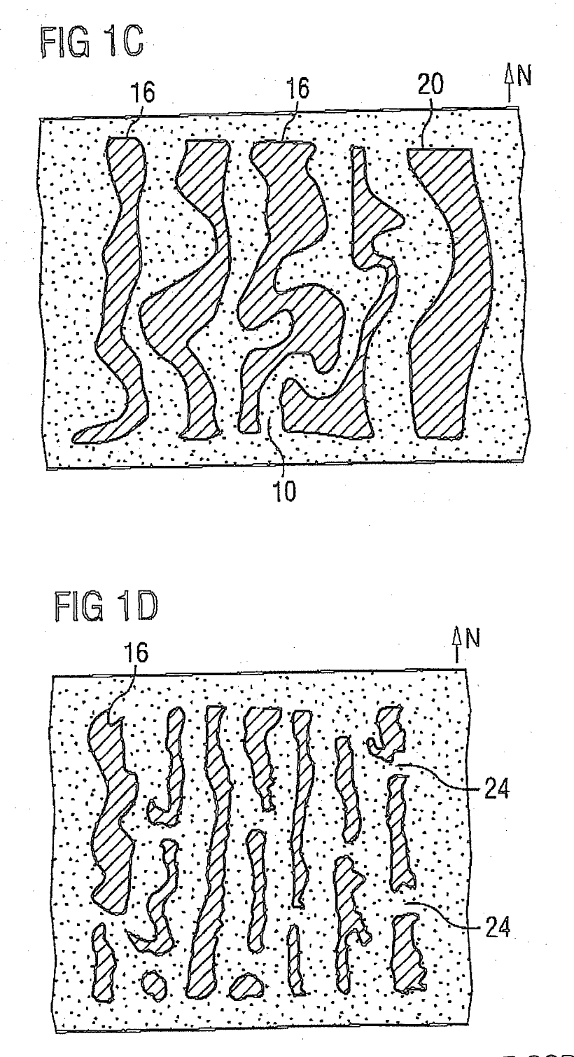

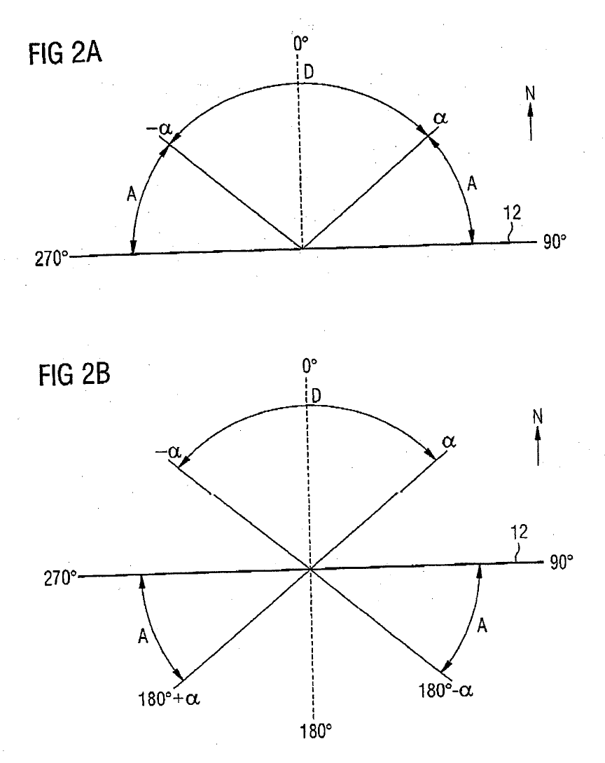

FIG. 2 shows diagrammatically excitation and detection ray paths which can be used particularly advantageously in connection with a detection method of the invention with reduced refractive index difference. FIG. 2(A) shows in a diagrammatic view the course of the ray path for a preferred incident light dark-field excitation. The surface 12 of the support structure which is designed to be two-dimensional is depicted as reference plane together with its normal direction N. All indicated angles are shown relative to the normal direction N, with a clockwise angular deflection being measured as positive. Thus an angle α=0° corresponds to the normal direction N. In the incident light dark-field excitation depicted in FIG. 2(A), the excitation of fluorescence light takes place in the excitation angle ranges A which extend from α-90° or 270° to −α. The excitation in this case may be carried out in one or in both excitation angle ranges A. The detection ray path is physically separated from the excitation ray path since only fluorescence light is collected in a detection angle range D from −α to α, in particular at 0°.

FIG. 2(B) shows an alternative excitation ray path. In this variant, which is referred to as transmitted light dark-field excitation, the excitation of fluorescence light takes place in an excitation angle range A between 90° and 180°−α or between 180°+α and 270°. Once again, one or both of the excitation angle ranges A can be chosen. Detection is again chosen in a detection angle range D between −α and α and is thus in the “dark field” of the excitation angle ranges A.

The lateral irradiation of the two-dimensional support structure with excitation light and collection of the fluorescence signal perpendicular to the support structure over an aperture angle range such that no direct light from the excitation ray path can enter the lens has, in combination with the leveling of the refractive index difference between pore wall and liquid, considerable advantages in relation to the detection sensitivity which can be achieved. The presented variants of incident light and transmitted light dark-field excitation can be implemented for example with an optical microscope with appropriate equipment (fluorescence filters, dark-field objectives, dark-field condenser, source of illumination such as mercury vapor lamp and LEDs). A laser can likewise serve as source of illumination. Köhler illumination of the support structure is preferred. However, other types of illumination such as, for example, so-called critical illumination can also be chosen. Reference is made in this connection to the statements in “Principles of Optics” by Max Born and Emil Wolf, 7th edition, Cambridge University Press, pages 595-599, which in this regard form an integral part of the disclosure of the present application.

The preferred dark-field excitation results in considerable advantages for sensitivity compared with conventional experimental constructions for reading fluorescence signals from microarrays, such as, for example, imaging fluorescence microscopy and laser scanning microscopy. In conventional experimental constructions, the excitation ray path and detection ray path run in parallel. In most cases, both ray paths use the same lens system in order to illuminate the support structure and collect the fluorescence light. It is correspondingly necessary to use optical filter systems for effective separation of excitation light and fluorescence light from one another. However, due to the high intensity of the excitation light, with these conventional experimental constructions, part of the excitation light, which is reflected and scattered from the microarray substrate, always reaches the detector through the support system. This proportion increases the background signal and thus makes the fluorescence signal-to-background ratio worse.

The “dark-field construction” according to the invention results in a physical separation between the excitation ray path and the detection ray path. The detection ray path is located in the so-called dark field of the excitation ray path, so that no excitation light can reach the detection ray path directly.

What is claimed is:

1. A method for detecting chemical and/or biochemical reactions and/or bindings, comprising the steps of:
providing a two-dimensional support structure which has, distributed over at least one surface region, a multiplicity of pores which extend continuously from one surface of the support structure to the opposite surface, wherein the pores are bounded by pore boundary areas of pore walls formed in the support structure, and the pore walls have a refractive index n_{pore wall} at a wavelength λ;
introducing a liquid into at least one of the pores of the support structure, where the refractive index n_{liquid} of the liquid at the wavelength λ is 0.90×n_{pore wall}≦n_{liquid}≦1.10×n_{pore wall};
coupling out light of a substance to be investigated from the at least one pore; and
detecting the light of the substance to be investigated.

2. The method as claimed in claim 1, wherein the light from the substance to be investigated is a light signal generated by absorption or reflection of excitation light, a luminescence light signal and/or a chemoluminescence light signal.

3. The method as claimed in claim 1, wherein the refractive index n_{liquid} of the liquid at the wavelength λ is 0.95×n_{pore wall}≦n_{liquid}≦1.05×n_{pore wall}.

4. The method as claimed in claim 1, wherein the refractive index n_{liquid} of the liquid at the wavelength λ is 0.99×n_{pore wall}≦n_{liquid}≦1.01×n_{pore wall}.

5. The method as claimed in claim 1, wherein capture molecules are immobilized via linker molecules on at least one of the pore boundary areas.

6. The method as claimed in claim 5, wherein the capture molecules are selected from the group consisting of DNA, proteins, and ligands.

7. The method as claimed in claim 5, wherein the capture molecules are oligonucleotide probes which are bound via terminal amino or thiol groups to linker molecules which in turn are bound via covalent and/or ionic group to the pore boundary area.

8. The method as claimed in claim 1, wherein the support structure comprises silicon, silica, and/or alumina.

9. The method as claimed in claim 1, wherein the liquid comprises polyethylene glycol.

10. The method as claimed in claim 9, wherein the liquid comprises a mixture of at least two polyethylene glycols with different chain lengths.

11. The method as claimed in claim 1, wherein the liquid comprises water-soluble carbohydrates and derivatives thereof, and the refractive index n_{liquid} can be adjusted via the concentration of the water-soluble carbohydrates.

12. The method as claimed in claim 1, further comprising the steps of:
exciting fluorescence light of the substance to be investigated by illuminating the substance to be investigated in the at least one pore along an excitation ray path with which excitation light impinges on the support structure, essentially at a predetermined excitation angle range from α to 180°−α and/or 180°+α to −α measured from a direction normal to the two-dimensional support structure; and
detecting the excited fluorescence light of the substance to be investigated along a detection ray path with which fluorescence light is detected essentially at a predetermined detection angle range from −α to α and/or 180°−α to 180°+α measured from the normal direction.

13. The method as claimed in claim 12, wherein the predetermined excitation angle range is from α to 90° and/or 270° to 270°+α.

14. The method as claimed in claim 12, wherein the predetermined excitation angle range is from 90° to 180°−α and/or 180°+α to 270°.

15. The method as claimed in claim 1, further comprising the step of introducing the substance to be investigated into the at least one pore before the step of introducing the liquid.

16. The method as claimed in claim 15, wherein the step of introducing the substance to be investigated comprises the steps of:
introducing the substance to be investigated into the at least one pore by means of a substance liquid comprising the substance to be investigated;
at least partly binding the substance to be investigated to capture molecules on the pore boundary areas; and
removing the substance liquid from the at least one pore.

17. The method as claimed in claim 1, further comprising the step of introducing the substance to be investigated into the at least one pore at the same time as performing the step of introducing the liquid.

18. A apparatus for detecting chemical and/or biochemical reactions and/or bindings, comprising:
a two-dimensional support structure which has, distributed over at least one surface region, a multiplicity of pores which extend continuously from one surface of the support structure to the opposite surface, wherein the pores are bounded by pore boundary areas of pore walls formed in the support structure, and the pore walls have a refractive index n_{pore wall} at a wavelength λ;
a liquid introduced into at least one of the pores of the support structure, where the refractive index n_{liquid} of the liquid at the wavelength λ is 0.90×n_{pore wall}≦n_{liquid}≦1.10×n_{pore wall};
a coupler that for coupling out light of a substance to be investigated from the at least one pore; and
a detector that detects the light of the substance to be investigated.

19. The apparatus as claimed in claim 18, wherein the light from the substance to be investigated is a light signal generated by absorption or reflection of excitation light, a luminescence light signal and/or a chemoluminescence light signal.

20. The apparatus as claimed in claim 18, wherein the refractive index n_{liquid} of the liquid at the wavelength λ is 0.95×n_{pore wall}≦n_{liquid}≦1.05×n_{pore wall}.

21. The apparatus as claimed in claim 18, wherein the refractive index n_{liquid} of the liquid at the wavelength λ is 0.99×n_{pore wall}≦n_{liquid}≦1.01×n_{pore wall}.

22. The apparatus as claimed in claim 18, further comprising capture molecules immobilized via linker molecules on at least one of the pore boundary areas.

23. The apparatus as claimed in claim 22, wherein the capture molecules are selected from the group consisting of DNA, proteins, and ligands.

24. The apparatus as claimed in claim 22, wherein the capture molecules are oligonucleotide probes which are bound via terminal amino or thiol groups to linker molecules which in turn are bound via covalent and/or ionic group to the pore boundary area.

25. The apparatus as claimed in claim 18, wherein the support structure comprises silicon, silica, and/or alumina.

26. The apparatus as claimed in claim 18, wherein the liquid comprises polyethylene glycol.

27. The apparatus as claimed in claim 26, wherein the liquid comprises a mixture of at least two polyethylene glycols with different chain lengths.

28. The apparatus as claimed in claim 18, wherein the liquid comprises water-soluble carbohydrates and derivatives thereof, and the refractive index n_{liquid} can be adjusted via the concentration of the water-soluble carbohydrates.

29. The apparatus as claimed in claim 18, further comprising an illuminator that excites fluorescence light of the substance to be investigated by illuminating the substance to be investigated in the at least one pore along an excitation ray path with which excitation light impinges on the support structure, essentially at a predetermined excitation angle range from α to 180°−α and/or 180°+α to −α measured from a direction normal to the two-dimensional support structure,
wherein the detector detects the excited fluorescence light of the substance to be investigated along a detection ray path with which fluorescence light is detected essentially at a predetermined detection angle range from −α to α and/or 180°−α to 180°+α measured from the normal direction.

30. The apparatus as claimed in claim 29, wherein the predetermined excitation angle range is from α to 90° and/or 270° to 270°+α.

31. The apparatus as claimed in claim 29, wherein the predetermined excitation angle range is from 90° to 180°−α and/or 180°+α to 270°.

32. The apparatus as claimed in claim 18, further comprising introducing means for introducing the substance to be investigated into the at least one pore before the step of introducing the liquid.

33. The apparatus as claimed in claim 32, wherein the introducing means for introducing the substance to be investigated, where the substance is introduced into the at least one pore by means of a substance liquid comprising the substance to be investigated, comprises:
means for at least partly binding the substance to be investigated to capture molecules on the pore boundary areas; and
removing means for removing the substance liquid from the at least one pore.

34. The apparatus as claimed in claim 18, further comprising introducing means for introducing the substance to be investigated into the at least one pore at the same time as introducing the liquid.

35. A apparatus for detecting chemical and/or biochemical reactions and/or bindings, comprising:
a two-dimensional support means which has, distributed over at least one surface region, a multiplicity of pores which extend continuously from one surface of the support structure to the opposite surface, wherein the pores are bounded by pore boundary areas of pore walls formed in the support structure, and the pore walls have a refractive index n_{pore wall} at a wavelength λ;
a liquid introduced into at least one of the pores of the support structure, where the refractive index n_{liquid} of the liquid at the wavelength λ is 0.90×n_{pore wall}≦n_{liquid}≦1.10×n_{pore wall};
coupling means for coupling out light of a substance to be investigated from the at least one pore; and
detecting means for detecting the light of the substance to be investigated.

36. The apparatus as claimed in claim 35, wherein the refractive index n_{liquid} of the liquid at the wavelength λ is 0.95×n_{pore wall}≦n_{liquid}≦1.05×n_{pore wall}.

37. The apparatus as claimed in claim 35, wherein the refractive index n_{liquid} of the liquid at the wavelength λ is 0.99×n_{pore wall}≦n_{liquid}≦1.01×n_{pore wall}.

38. The apparatus as claimed in claim 35, further comprising an illuminating means for exciting fluorescence light of the substance to be investigated by illuminating the substance to be investigated in the at least one pore along an excitation ray path with which excitation light impinges on the support structure, essentially at a predetermined excitation angle range from α to 180°−α and/or 180°+α to −α measured from a direction normal to the two-dimensional support structure,
wherein the detecting means detects the excited fluorescence light of the substance to be investigated along a detection ray path with which fluorescence light is detected essentially at a predetermined detection angle range from −α to α and/or 180°−α to 180°+α measured from the normal direction.

39. The apparatus as claimed in claim 35, further comprising introducing means for introducing the substance to be investigated into the at least one pore before the step of introducing the liquid.

40. The apparatus as claimed in claim 39, wherein the introducing means for introducing the substance to be investigated, where the substance is introduced into the at least one pore by means of a substance liquid comprising the substance to be investigated, comprises:
means for at least partly binding the substance to be investigated to capture molecules on the pore boundary areas; and
removing means for removing the substance liquid from the at least one pore.

41. The apparatus as claimed in claim 35, further comprising introducing means for introducing the substance to be investigated into the at least one pore at the same time as introducing the liquid.
